(19) United States
(12) Patent Application Publication
(10) Pub. No.: US 2014/0163653 A1
(43) Pub. Date: Jun. 12, 2014

(54) Epidermis Enhancing Assembly

(71) Applicant: Steven Cano, Henderson, NV (US)

(72) Inventor: Steven Cano, Henderson, NV (US)

(21) Appl. No.: 13/710,422

(22) Filed: Dec. 10, 2012

(51) Int. Cl. A61F 7/00

(52) U.S. Cl. CPC A61F 7/00
USPC 607/109

(57) ABSTRACT

An epidermis enhancing assembly that enhances the epidermis through a multi-step cleansing process that cleanses the epidermal area without excessive drying. A control portion regulates the distribution of liquids and electrical functions of the assembly. The control portion includes a heated liquid container, a cool liquid container, and a reservoir. Pumps disperse a heated liquid onto the epidermis for initial cleaning and opening of the pores. A carrier portion transports the liquids to and from the control portion. An epidermal engagement device joins with the carrier portion, and serves to engage the epidermis with bristles. The bristles vibrate in a manner efficacious for manipulating the pores and the epidermis. The pumps then force a cool liquid onto the epidermis for closing the pores and further cleansing. A vacuum then sucks the liquids back to the assembly after cleansing is complete.

### CROSS-REFERENCE TO RELATED APPLICATIONS

The present Utility patent application claims priority benefit of the [U.S. provisional application for Patent Ser. No. 61/630,435 entitled “DermXtract System”, filed on Dec. 13, 2011 under 35 U.S.C. 119(e). The contents of this related provisional application are incorporated herein by reference for all purposes to the extent that such subject matter is not inconsistent herewith or limiting hereof.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to the epidermis. More particularly, the invention relates to epidermal enhancement.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that the epidermis is composed of the outermost layers of cells in the skin. The ability of the skin to hold water is primarily due to the stratum corneum and is critical for maintaining healthy skin.

Typically, a cleanser is a product used to cleanse something, such as a detergent or industrial cleaning agent. A popular type of cleanser is a facial care product that is used to remove make-up, dead skin cells, oil, dirt, and other types of pollutants from the skin of the face. This helps to unclog pores and prevent skin conditions such as acne.

Typically, a cleanser may be utilized once or more times a day as part of a skin care regimen together with a toner and a moisturizer. Using a cleanser to remove dirt is considered to be a better alternative to bar soap or another form of skin cleanser not specifically formulated for the skin.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a detailed perspective view of an exemplary epidermis enhancing assembly, in accordance with an embodiment of the present invention;

FIG. 2 illustrates a detailed perspective view of an exemplary at least one container, in accordance with an embodiment of the present invention;

FIG. 3 illustrates a sectioned view of an exemplary control portion, in accordance with an embodiment of the present invention;

FIG. 4 illustrates a close up views of an exemplary carrier portion, in accordance with an embodiment of the present invention;

FIGS. 5A and 5B illustrate detailed perspective views of an exemplary epidermal engagement device, in accordance with an embodiment of the present invention, where FIG. 5A illustrates a frontal view of an exemplary epidermal engagement device, and FIG. 5B illustrates a back view of an exemplary epidermal engagement device; and

FIGS. 6A and 6B illustrate detailed perspective views of an exemplary plurality of bristles, in accordance with an embodiment of the present invention, where FIG. 6A illustrates the plurality of bristles in a level orientation, and FIG. 6B illustrates the plurality of bristles in an angled orientation.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

There are various types of epidermis enhancing assemblies that may be provided by preferred embodiments of the present invention. In one embodiment of the present invention, the epidermis enhancing assembly may enhance the epidermis by removing dermatological conditions from an epidermal area. The dermatological conditions may include, without limitation, epidermal residue, acne, dirt, oil, cosmetics, prominent acne scars, prominent wrinkles, loose skin, dermatological infections, and simple cosmetic blemishes. In some embodiments, the epidermis enhancing assembly may enhance the epidermis through a multi-step cleansing process that cleanses the epidermal area without excessive drying. The cleansing process may include engaging an epidermal area with a heated liquid operable to open a plurality of pores on the epidermal area. An epidermal engagement device may then engage the epidermal area with a plurality of bristles that vibrate for cleansing and exfoliating functionality. In some embodiments, a cool liquid may then disperse onto the epidermal area for closing the plurality of pores and further cleansing. The liquids may recirculate back to the epidermis enhancing assembly after completion.

In one embodiment of the present invention, the epidermis enhancing assembly may utilize liquids at various temperatures to manipulate a plurality of pores in the epidermis. Those skilled in the art, in light of the present teachings, will recognize that a heated liquid may cause a plurality of pores in the epidermis to open, while conversely, a cool liquid may close the plurality of pores. The epidermis enhancing assembly may include a control portion for regulating various components of the epidermis enhancing assembly. The control portion may be operable to regulate temperature of liquids, provide electrical power, provide a pump for dispersing liquids, and provide a vacuum for returning liquids. In some embodiments, the control portion may include at least one container configured to store a heated liquid and a cool liquid. The at least one container may include a heating portion and a cooling portion. The heating portion may be configured to contain a heated liquid. The cooling portion may be configured to contain a cool liquid. In some embodiments, at least one pump may force the respective liquid from either the cool portion or the heated portion to the epidermal area. A reservoir portion may be configured to recapture the heated liquid or the cool liquid after use on the epidermal area. The reservoir portion may join with a vacuum that is operable to force the respective liquid into the reservoir portion.

In one embodiment of the present invention, an epidermal engagement device may be configured to be operable to engage an epidermal area for cleaning and exfoliation. The epidermal engagement device may be operable to disperse either the heated liquid or the cool liquid on an epidermal area. The epidermal engagement device may further serve to recapture either liquid back to the reservoir portion from the epidermal area. In some embodiments, the epidermal engagement device may include a carrier portion for transporting liquids from the heating portion, cooling portion, or reservoir portion to and from the epidermal area. The carrier portion may include a proximal end that joins with the control portion. The carrier portion may further include a distal end that is operable to engage the epidermal area. The distal end may join with a plurality of bristles that are configured to be operable to engage a plurality of pores in the epidermal area. In one embodiment, the plurality of bristles may be configured to be operable to manipulate the plurality of pores for enhancing the cleaning process. In some embodiments, the plurality of bristles may be operable to vibrate while engaging the epidermal area for providing a more thorough engagement of the plurality of pores in the epidermal area. In some embodiments, the carrier portion may include a dispersion carrier for transporting the liquids from the at least one container to the epidermal area. The carrier portion may further include a return carrier for transporting the liquids from the epidermal area to the reservoir portion. The carrier portion may further include an electrical carrier for transporting electricity to the plurality of bristles while vibrating.

In one embodiment of the present invention, at least one pump may be operable to disperse the heated liquid onto the epidermal area, thereby helping to open the plurality of pores. In this manner, the plurality of bristles may engage inside the plurality of pores more thoroughly. In some embodiments, the plurality of bristles may vibrate and be angled to provide enhanced reach inside the plurality of pores. In another embodiment, the plurality of bristles may extend and retract from the distal end for enhanced cleaning In some embodiments, the at least one pump may disperse the cool liquid onto the epidermal area for helping to close the pores, whereby the plurality of bristles may continue exfoliating and cleaning the surface of the epidermal area even after the plurality of pores close. In some embodiments, a vacuum may force the liquids through the return carrier, whereby both the heated liquid and the cooler liquid, along with any epidermal residue, recirculate back to the at least one liquid reservoir for storage. Those skilled in the art, in light of the present teachings, will recognize that the simultaneous combination of warmed liquids, mild vibrations, cool liquids, and suction may stimulate and cleanse the epidermal area. Additional benefits may include, without limitation, blood circulation, color enhancement, surgery preparation.

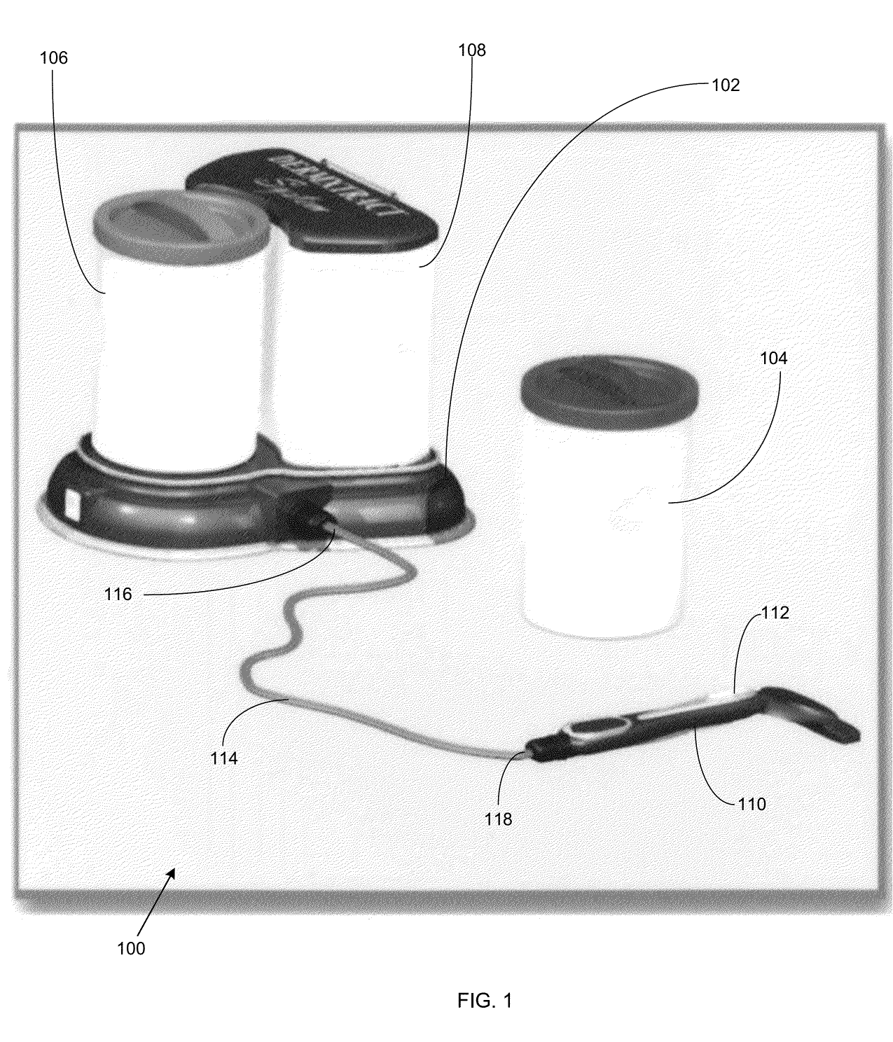

FIG. 1 illustrates a detailed perspective view of an exemplary epidermis enhancing assembly, in accordance with an embodiment of the present invention. In the present invention, the epidermis enhancing assembly 100 may enhance the epidermis by removing dermatological conditions from an epidermal area. The dermatological conditions may include, without limitation, epidermal residue, acne, dirt, oil, cosmetics, prominent acne scars, prominent wrinkles, loose skin, dermatological infections, and simple cosmetic blemishes. Those skilled in the art, in light of the present teachings, will recognize that properly cleansing the epidermal area may provide effective cleansing treatment to erysipelas, impetigo, general dermatitis and staphylococcal infections. In some embodiments, the epidermis enhancing assembly may enhance the epidermis through a multi-step cleansing process that cleanses the epidermal area without excessive drying. The cleansing process may include engaging an epidermal area with a heated liquid operable to open a plurality of pores on the epidermal area. An epidermal engagement device may then engage the epidermal area with a plurality of bristles that vibrate for cleansing and exfoliating functionality. In some embodiments, a cool liquid may then disperse onto the epidermal area for closing the plurality of pores and further cleansing. The liquids may recirculate back to the epidermis enhancing assembly after completion.

In one embodiment of the present invention, the epidermis enhancing assembly may utilize liquids at various temperatures to manipulate a plurality of pores in the epidermis. The liquids may include, without limitation, water, cleansing solutions, natural extracts, astringents, and all other human antibacterial compositions. Those skilled in the art, in light of the present teachings, will recognize that a heated liquid may cause a plurality of pores in the epidermis to open, while conversely, a cool liquid may close the plurality of pores. The epidermis enhancing assembly may include a control portion 102 for regulating various components of the epidermis enhancing assembly. The control portion may be operable to regulate temperature of liquids, provide electrical power, provide a pump for dispersing liquids, and provide a vacuum for returning liquids. In one embodiment, the control portion may be fabricated from high-impact polystyrene. The control portion may be dimensioned about 8″×4″×4″. However, in other embodiments, the control portion may be sized larger or smaller. However, in other embodiments, the base and each containers may be smaller or larger to accommodate various activities, including, without limitation, traveling, sports, salons, and commercial embodiments.

In some embodiments, the control portion may include at least one container configured to store a heated liquid and a cool liquid. In one embodiment, the control portion may include an eclectic assortment of attachments efficacious for enhancing the epidermis. The control portion may adapt and interchange with various attachments, including, without limitation, tubing, power wire, extensions, and topical ointment containers. In this manner, different attachments may provide the assembly with additional functions. In some embodiments, the at least one container may include a heating portion 104 configured to contain the heated liquid, and a cooling portion 106 configured to contain the cool liquid. The heating portion and the cooling portion may be sized and dimensioned at a maximum height of 3″ and retain five ounces of liquid. In some embodiments, each container may be fabricated from polyvinyl chloride. A reservoir portion 108 may be configured to recapture the heated liquid or the cool liquid after use. In some embodiments, the reservoir container may be sized and dimensioned at eleven ounces. However, larger or smaller sizes may be possible. In some embodiments, the bottom plane of the control portion may include an internal vacuum directly below the reservoir portion, and at least one pump in direct alignment on the opposite side of the heating container and the cooling container. The at least one pump may force the respective liquids from either the cool portion or the heated portion. The reservoir portion may join with the vacuum that is operable to force the respective liquid into the reservoir portion. Suitable materials for fabricating the epidermis enhancing assembly may include, without limitation, high-impact polystyrene (HIPS), high-density polyethylene (HDPE), rigid polyvinyl chloride (r-PVC), polyamide, styrene-butadiene rubbers, plastic, fiberglass, and metal.

In one embodiment of the present invention, an epidermal engagement device 110 may be configured to be operable to engage an epidermal area for cleaning and exfoliation. The epidermal engagement device may be operable to disperse either the heated liquid or the cool liquid on an epidermal area. At least one switch 112 may regulate the flow of each liquid and additional electrical function for the epidermal engagement device. The epidermal engagement device may further serve to recapture either liquid back to the reservoir portion from the epidermal area. In some embodiments, the epidermal enhancement assembly may include a carrier portion 114 for transporting liquids from the heating portion, cooling portion, or reservoir portion to and from the epidermal area. The carrier portion may include a proximal end 116 that joins with the control portion. The carrier portion may further include a distal end 118 that is operable to engage the epidermal area. The distal end may join with a plurality of bristles. The plurality of bristles may be configured to be operable to engage a plurality of pores inside the epidermal area. In some embodiments, the plurality of bristles may be operable to vibrate while engaging the epidermal area for providing a more thorough engagement of the plurality of pores in the epidermal area. In some embodiments, the carrier portion may include a dispersion carrier for transporting the liquids from the at least one container to the epidermal area. The carrier portion may further include a return carrier for transporting the liquids from the epidermal area to the reservoir portion. The carrier portion may further include an electrical carrier for transporting electricity to the plurality of bristles while vibrating.

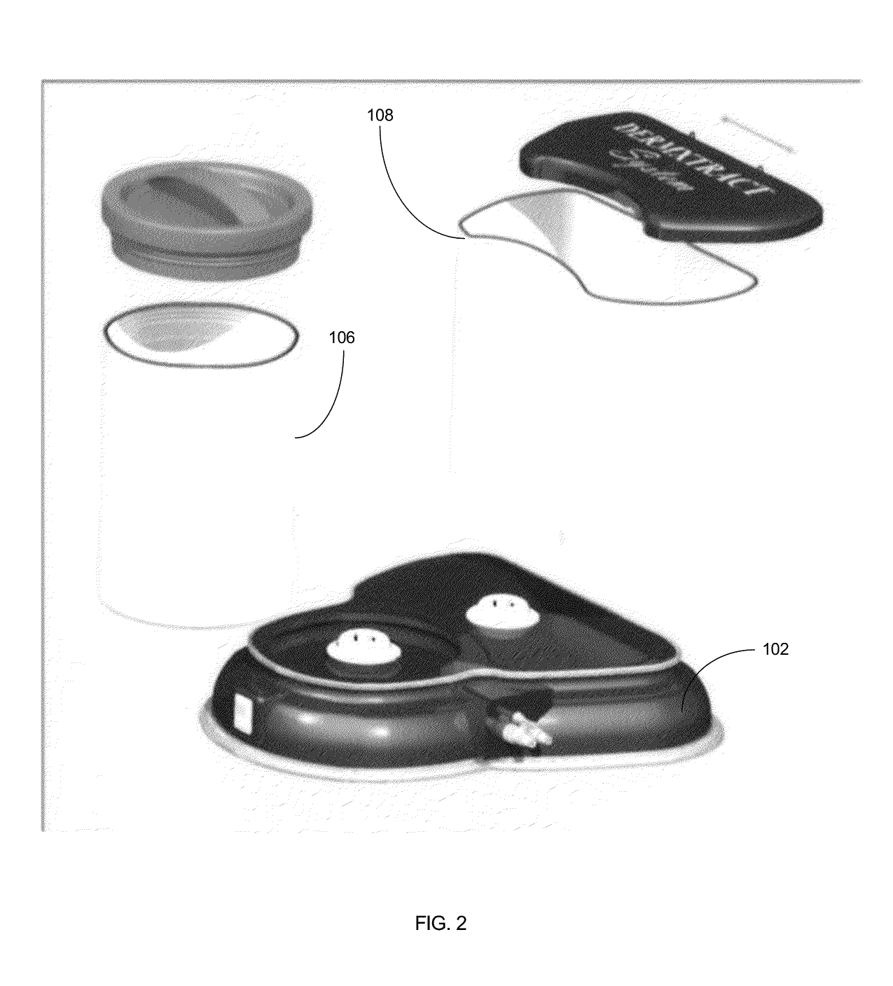

FIG. 2 illustrates a detailed perspective view of an exemplary at least one container, in accordance with an embodiment of the present invention. In the present invention, at least one pump may be operable to disperse the heated liquid onto the epidermal area, thereby helping to open the plurality of pores. In this manner, the plurality of bristles may engage inside the plurality of pores more thoroughly. In some embodiments, the plurality of bristles may vibrate and be angled to provide enhanced reach inside the plurality of pores. In another embodiment, the plurality of bristles may extend and retract from the distal end for enhanced cleaning In some embodiments, the at least one pump may disperse the cool liquid onto the epidermal area for helping to close the pores, whereby the plurality of bristles may continue exfoliating and cleaning the surface of the epidermal area even after the plurality of pores close. In some embodiments, a vacuum may force the liquids through the return carrier, whereby both the heated liquid and the cooler liquid, along with any epidermal residue, recirculate back to the at least one liquid reservoir for storage.

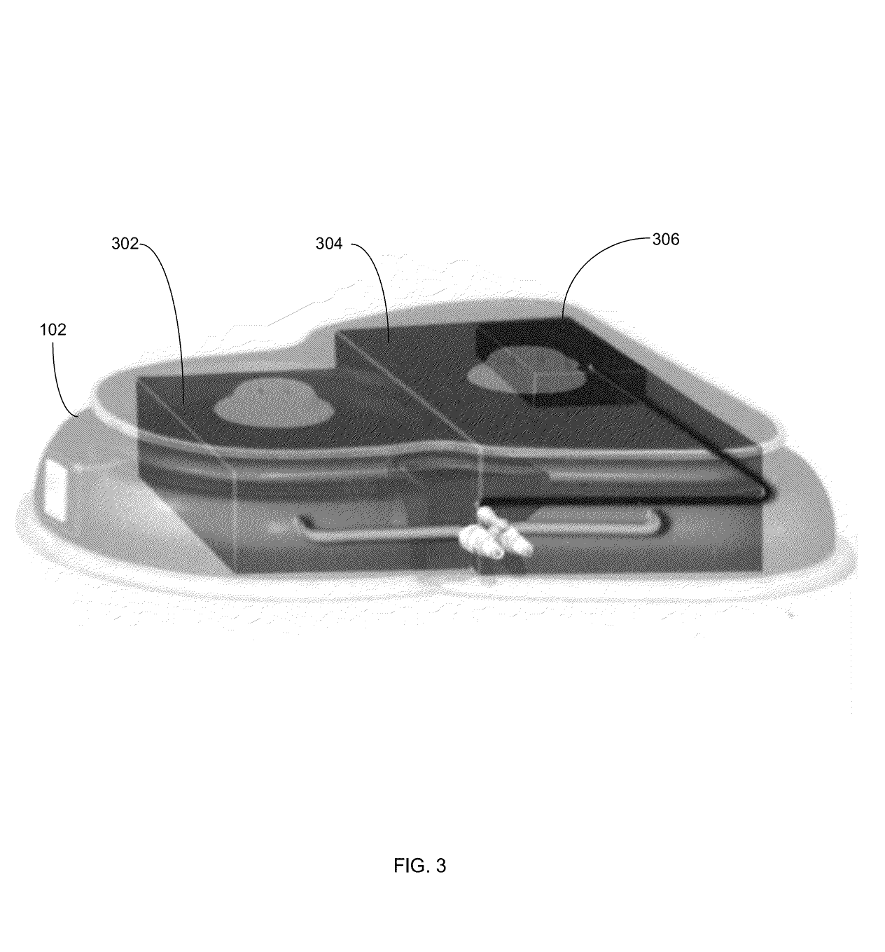

FIG. 3 illustrates a sectioned view of an exemplary control portion, in accordance with an embodiment of the present invention. In the present invention, the control portion may be configured in variations in which the at least one container for the heated liquid and the cool liquid may be hosted within the unit simultaneously. In this manner, the heating portion and the cooling portion may have at least one pump 302 for dispersing the respective liquid. However in another embodiment, a single pump may regulate the liquid in the heating portion and the cooling portion. The at least one pump may include an approximate one-quarter horsepower capacity for an approximate production of one cubic feet per minute and pressure rating of about 20 pounds per square inch. A connecting module may extend from the control portion to the at least one pump. In some embodiments, the bottom plane of the control portion may include an internal vacuum 304 directly below the reservoir portion. The reservoir portion may join with the vacuum that is operable to force the respective liquid into the reservoir portion from the epidermal area.

In some embodiments, the control portion may be configured in variations capable of independently heating and/or cooling the contents of its respective container. In such variations, the control portion may achieve such temperatures in various methods, including but not limited to thermoelectric sleeves in proximity to the hosting compartments of the at least one container, and thermoplates positioned in the flooring of the hosting compartments upon the bottom exteriors of the at least one container. In such variations, the control portion may provide both heating and cooling of the at least one container. However in another embodiment, the control portion may only accommodate one of such temperature-controlling methods. In one alternative embodiment, the at least one container may include a separate container for storage of medicinal and/or cosmetic liquids, including, but not limited to, cleansers, astringents and moisturizers. In such variations, the control portion may include a third port in its base for hosting of the medicinal container. In some embodiments, the control portion may include a power source 306. The power source may include, without limitation, a battery, an external power source, and a solar panel. The power source may be powered by alternating current exclusively through power cord. The control portion may include various colors, and may or may not bear a logo, emblem, or specific design, which may or may not be a registered trademark and/or copyright.

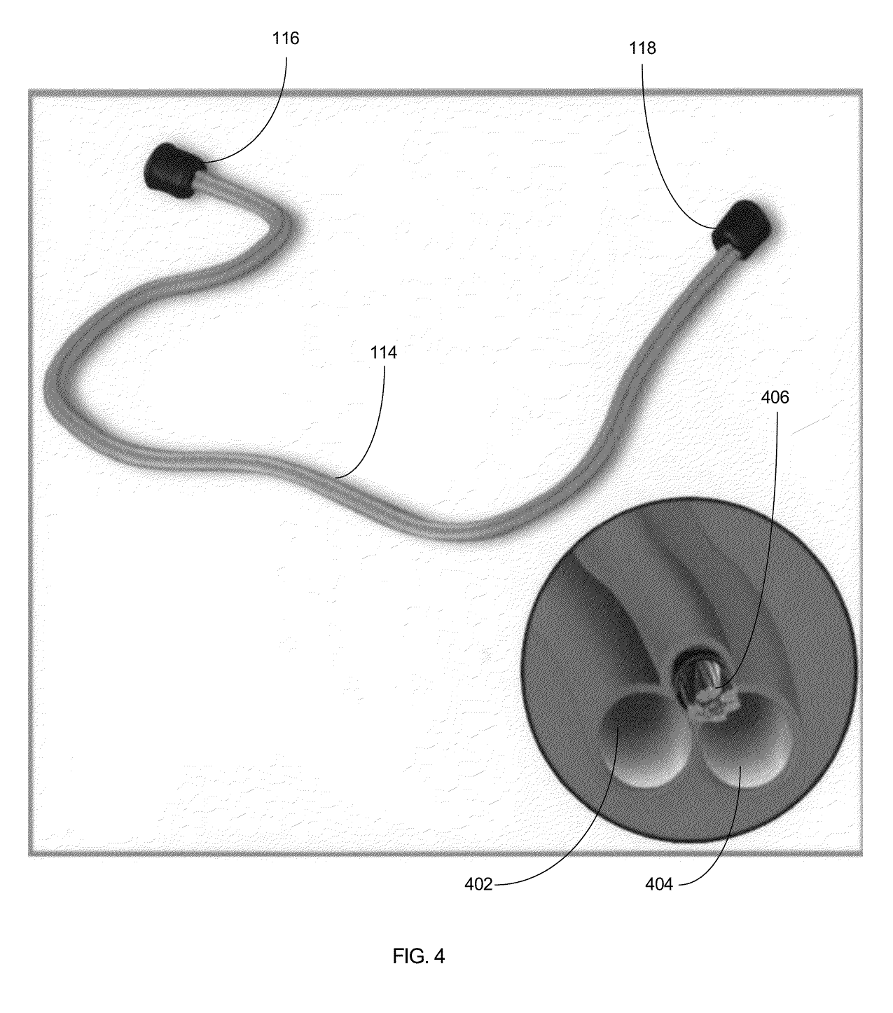

FIG. 4 illustrates a close up views of an exemplary carrier portion, in accordance with an embodiment of the present invention. In the present invention, the carrier portion may join the epidermal enhancing device to the control portion. The carrier portion may include a tube and may be of various lengths. In some embodiments, the carrier portion may include a dispersion carrier 402 for transporting the liquids from the at least one container to the epidermal area. The carrier portion may further include a return carrier 404 for transporting the liquids from the epidermal area to the reservoir portion. The carrier portion may further include an electrical carrier 406 for transporting electricity to the plurality of bristles while vibrating. In one alternative embodiment, a medicinal carrier may transport a topical ointment from the at least one container to the epidermal area. Suitable materials for the carrier portion may include, without limitation, plasticized polyvinyl chloride (p-PVC), low-density polyethylene (LDPE), plastic, fiberglass, and styrene-butadiene rubbers.

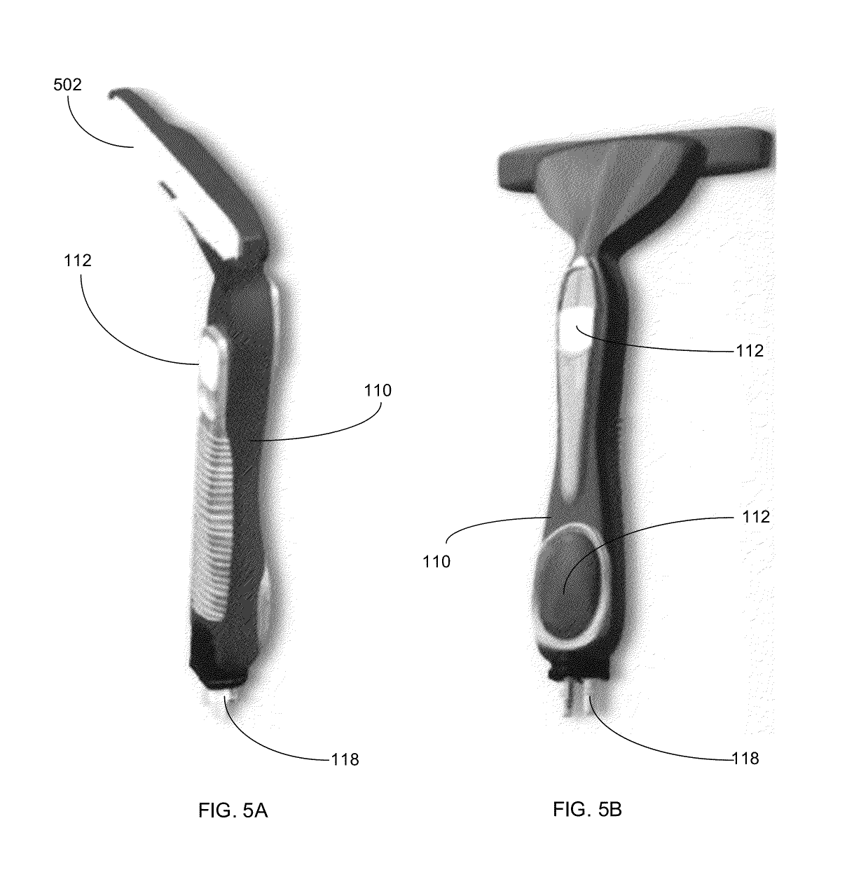

FIGS. 5A and 5B illustrate detailed perspective views of an exemplary epidermal engagement device, in accordance with an embodiment of the present invention, where FIG. 5A illustrates a frontal view of an exemplary epidermal engagement device, and FIG. 5B illustrates a back view of an exemplary epidermal engagement device. In the present invention, the epidermal engagement device may be individually controllable in power activation and/or speed of operation. However, in some embodiments, the epidermal engagement device may not be operable to vibrate a plurality of bristles 502 for more thorough cleansing of the epidermal area. Those skilled in the art, in light of the present teachings, will recognize that the epidermal engagement device may be shaped and dimensioned for strategic application upon various parts of the face, including, without limitation, a small circular head for application about the temples and hairline, and a straight column for use in removal of nasal sebum from the lateral creases of the nose.

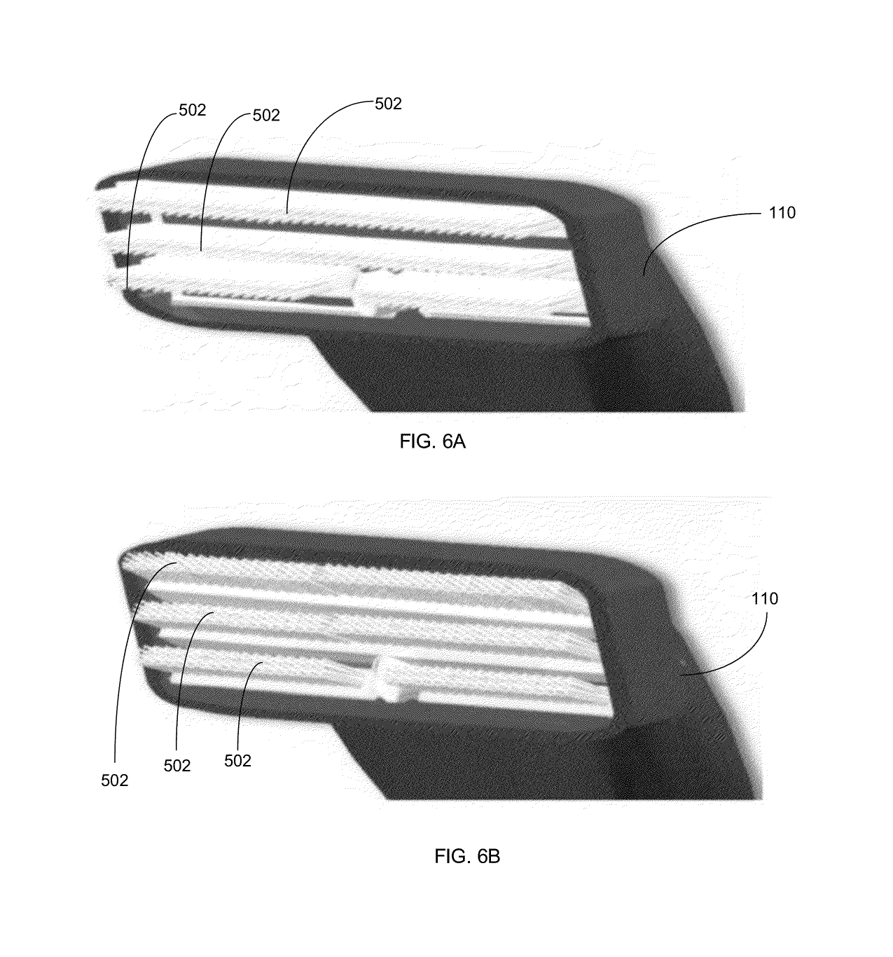

FIGS. 6A and 6B illustrate detailed perspective views of an exemplary plurality of bristles, in accordance with an embodiment of the present invention, where FIG. 6A illustrates the plurality of bristles in a level orientation, and FIG. 6B illustrates the plurality of bristles in an angled orientation. In the present invention, the plurality of bristles may extend from a T-shaped wand. The wand may include a handle sized at about 4″ length, and a crossbar of which measures about 2″ in width. A switch portion may position on the handle to regulate the at least one pump. In some embodiments, an external surface of the crossbar, in proximity to the perimeter, a plurality of bristles may extend. The plurality of bristles may be fabricated of a polyamide material and be sized ¼″ long. The plurality of bristles may position upon a track plane that attaches to the external facing of the crossbar. The track plane may include a lever that extends through the wand to adjust the orientation of the plurality of bristles from ninety degrees to forty-five degrees. In this manner, the plurality of bristles may position at an even orientation and an angled orientation for enhanced engagement with the epidermal area and the plurality of pores. In some embodiments, the plurality of bristles may include various lengths and diameters, and may be featured at various areas upon the endpoint of the wand. In some embodiments, the plurality of bristles may be positioned in various patterns, and be set at various angles. In some embodiments, the plurality of bristles may or may not be empowered to rotate in circular patterns. In such variations, a various number of bristle rings may be included, which may or may not rotate individually and in contrasting clockwise and counterclockwise patterns.

In some embodiments, the epidermal engagement device may include an eclectic assortment of wands that vary significantly from a T-shaped wand. Those skilled in the art, in light of the present teachings, will recognize that myriad shapes, sizes, applications, and utilities for the epidermal engagement device may join with the carrier portion. For example, without limitation, a wand configured with a flat fan spray nozzle may join with tubing to deliver the appropriate liquid and an ointment to the epidermal area. Additional epidermal engagement devices may include, without limitation, full cone spray nozzles, half cone spray nozzles, uni spray nozzles, quick disconnect nozzles, adjustable ball type nozzles, clamp eyelet nozzles, spiral spray nozzles, liquid mixing jet educator, and tank washing nozzles. In such variations, the functions and techniques for engaging the epidermal area may allow the epidermal engagement device to perform additional functions, including, without limitation, a massager, a heater, a shaver, hair and scalp washing, dead skin and bunion removal, full body cleansing, face mask instant skin cleansing.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing epidermal cleaning and exfoliation with minimal drying according to the present invention will be apparent to those skilled in the art. Various aspects of the invention have been described above by way of illustration, and the specific embodiments disclosed are not intended to limit the invention to the particular forms disclosed. The particular implementation of the epidermal cleaning and exfoliation with minimal drying may vary depending upon the particular context or application. By way of example, and not limitation, the epidermal cleaning and exfoliation with minimal drying described in the foregoing were principally directed to applying warm liquids to open pores for engagement with vibrating bristles, followed by cool water to close the pores again implementations; however, similar techniques may instead be applied to animal epidermal care, where the bristles may be stronger, and the liquid may include harsh detergents, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. An assembly comprising:
a control portion, said control portion comprising a heating portion, said heating portion being configured to contain a heated liquid, said control portion further comprising a cooling portion, said cooling portion being configured to contain a cool liquid, said control portion further comprising a reservoir portion, said reservoir portion being configured to receive said heated liquid and said cool liquid;
an epidermal engagement device, said epidermal engagement device being configured to be operable to enhance an epidermal area, said epidermal engagement device comprising a plurality of bristles, said plurality of bristles being configured to be operable to engage said epidermal area; and
a carrier portion, said carrier portion being configured to transport said heated liquid to and from said control portion and said epidermal area, said carrier portion further being configured to transport said cool liquid to and from said control portion and said epidermal area, said carrier portion further being configured to transport electricity from said control portion to said plurality of bristles.

2. The assembly of claim 1, in which said assembly comprises an epidermis enhancing assembly.

3. The assembly of claim 1, wherein said control portion is operable to provide heat to said heating portion with a thermoelectric sleeve.

4. The assembly of claim 1, in which said control portion comprises at least one pump, said at least one pump being operable to disperse said heated liquid and said cool liquid.

5. The assembly of claim 1, in which said control portion comprises a vacuum, said vacuum being operable to return said heated liquid and said cool liquid to said reservoir portion.

6. The assembly of claim 1, in which said control portion comprises a power source.

7. The assembly of claim 1, in which said liquid comprises water.

8. The assembly of claim 1, in which said epidermal engagement device comprises a T-shaped wand.

9. The assembly of claim 8, wherein said plurality of bristles is configured to extend from said T-shaped wand.

10. The assembly of claim 1, wherein said plurality of bristles are operable to position in a level orientation and an angled orientation.

11. The assembly of claim 1, wherein said plurality of bristles are operable to vibrate.

12. The assembly of claim 1, in which carrier portion comprises a tube.

13. The assembly of claim 1, in which said carrier portion comprises a proximal end, said proximal end being disposed to join with said control portion.

14. The assembly of claim 1, in which said carrier portion comprises a distal end, said proximal end being disposed to join with said plurality of bristles.

15. The assembly of claim 1, in which carrier portion comprises a dispersion carrier, said dispersion carrier being configured to transport said heated liquid from said heated portion to said epidermal area.

16. The assembly of claim 1, in which carrier portion comprises a dispersion carrier, said dispersion carrier being configured to transport said cool liquid from said cooling portion to said epidermal area.

17. The assembly of claim 1, in which carrier portion comprises a return carrier said return carrier being configured to transport said heated liquid and said cool liquid from said epidermal area to said reservoir portion.

18. The assembly of claim 1, in which carrier portion comprises an electrical carrier.

19. An assembly comprising:
means for filling a heating portion and a cooling portion with a liquid;
means for applying a topical ointment on an epidermal area;
means for dispersing a heated liquid to said epidermal area;
means for opening a plurality of pores;
means for engaging said epidermal area with an epidermal engagement device;
means for engaging said plurality of pores with a plurality of bristles;
means for positioning said plurality of bristles from a level orientation to an angles orientation;
means for dispersing a cool liquid to said epidermal area;
means for engaging said epidermal area with said epidermal engagement device; and
means for returning said heated liquid and said cool liquid from said epidermal area to a reservoir portion.

20. An assembly consisting of:
a control portion, said control portion comprising a heating portion, said control portion being operable to provide heat to said heating portion with a thermoelectric sleeve, said heating portion being configured to contain a heated liquid, said control portion further comprising a cooling portion, said cooling portion being configured to contain a cool liquid, said control portion further comprising a reservoir portion, said reservoir portion being configured to receive said heated liquid and said cool liquid, said control portion further comprising at least one pump, said at least one pump being operable to disperse said heated liquid and said cool liquid; said control portion further comprising a vacuum, said vacuum being operable to return said heated liquid and said cool liquid to said reservoir portion, said control portion further comprising a power source;
an epidermal engagement device, said epidermal engagement device being configured to be operable to enhance an epidermal area, said epidermal enhancement device comprising a T-shaped wand, said epidermal engagement device comprising a plurality of bristles, said plurality of bristles being configured to be operable to engage said epidermal area, said plurality of bristles being operable to position in a level orientation and an angled orientation, said plurality of bristles being operable to vibrate; and
a carrier portion, said carrier portion comprising a tube, said carrier portion further comprising a proximal end, said proximal end being disposed to join with said control portion, said carrier portion further comprising a distal end, said distal end being disposed to join with said epidermal engagement device, said carrier portion further comprising a dispersion carrier, said dispersion carrier being configured to transport said heated liquid and said cool liquid from said control portion to said epidermal area, said carrier portion further comprising a return carrier, said return carrier being configured to transport said heated liquid and said cool liquid from said epidermal area to said reservoir portion, said carrier portion further comprising an electrical carrier, said electrical carrier being configured to transport electricity from said control portion to said plurality of bristles.
